(19) United States
(12) Patent Application Publication
(10) Pub. No.: US 2013/0310841 A1
(43) Pub. Date: Nov. 21, 2013

(54) Femoral Sizing Devices and Procedures for Use in Knee Surgery

(71) Applicant: Wolfgang Fitz, Sherborn, MA (US)

(72) Inventor: Wolfgang Fitz, Sherborn, MA (US)

(73) Assignee: THE BRIGHAM AND WOMEN'S HOSPITAL, INC., Boston, MA (US)

(21) Appl. No.: 13/818,301

(22) PCT Filed: Aug. 24, 2011
(86) PCT No.: PCT/US11/49023

§ 371 Date: Feb. 21, 2013

Related U.S. Application Data
(60) Provisional application No. 61/377,255, filed on Aug. 26, 2010.

(51) Int. Cl. A61B 17/15; A61B 19/00

(52) U.S. Cl. CPC A61B 17/155; A61B 19/46
USPC 606/102

(57) ABSTRACT

The present invention is directed to a femoral sizing device that can be used in knee surgery and to techniques in which this device may prove useful.

### CROSS REFERENCE TO RELATED APPLICATIONS

The present application claims the benefit of U.S. provisional application 61/377,255 filed on Aug. 26, 2010, the contents of which is hereby incorporated by reference in its entirety.

### FIELD OF THE INVENTION

The present invention is directed to medical devices for determining parameters needed for the resection of bones, and particularly for the resection of the distal end of femoral bones during knee replacement surgery. In addition, the invention is directed to improved procedures for determining these parameters.

### BACKGROUND OF THE INVENTION

Total knee arthroplasty is one of the most successful orthopedic operations for relieving pain and restoring function. However, restoration of normal knee kinematics is not entirely possible with most current implants and total knee replacement (TKR) procedures (Dennis, et al., Clin. Orthop. 356:47 (1998); Stiehl, et al., J. Bone Joint Surg. Br. 77:884 (1995); Dennis, et al., Clin. Orthop. 428:180 (2004); Stiehl, Clin. Orthop. 365:139 (1999); Banks, et al., J. Arthroplasty 3:297 (1997); cf. Bertin, et al., J. Arthroplasty 17:1040 (2002)). Although many devices and techniques have been developed for use during knee surgery (see U.S. Pat. No. 5,624,444; U.S. Pat. No. 6,458,135; U.S. Pat. No. 6,106,529; U.S. Pat. No. 6,024,746; U.S. Pat. No. 6,173,200; U.S. Pat. No. 4,721,104; US 2007/0173851; WO 99/20192; Price, et al., J. Arthroplasty 16(8):970-976 (2001)) substantial improvements are still needed.

### SUMMARY OF THE INVENTION

The present invention is directed to a femoral sizing device for making measurements of the distal femur during knee surgery. The primary elements of the device are: a) a first gauge (in some embodiments, referred to herein as a central feeler gauge and shown in FIG. 1 as element (4)); and b) a femoral sizing guide (shown in FIG. 1 as element (3)) which engages the first gauge and which includes means for attaching a spacer block. Attchment may be accomplished by screws, pins, clamps, clasps pegs or, most preferably, holes that are capable accepting pegs from the spacer block as shown in FIGS. 2, 5 and 7. The femoral sizing guide must also have means for positioning an attached spacer block beneath a postmedial condyle of a femur when the sizing guide engages the first gauge and the first gauge is suspended from the lowest point of the trochlear groove of the femur (see FIG. 5). The means may be provided by the shape of the femoral sizing guide or by a separate component attached to the guide. The most preferred means for positioning the spacer arm is by a fixed arm that is located at one end of a longitudinal member of the sizing guide and that extends in a direction away from this longitudinal member as shown in FIGS. 2 and 7.

When fully assembled and in use, the device will include: a) a first gauge that includes a trochlear attachment section (in some embodiments referred to as a transverse arm and shown in FIG. 3 as (16)) for suspending the gauge from the lowest point of the trochlear groove of the femur; b) a femoral sizing guide, comprising a main body which engages the first gauge; and c) at least one spacer block (typically 1-8 mm in thickness), attached to the lower portion of the femur sizing guide. Optionally, the device may also include one or more shims in contact with the top of the spacer block to increase its thickness and a second gauge which, unlike the first, does not have a transverse arm (in some embodiments, referred to herein as a lateral feeler gauge and shown in FIG. 1 as element (9)).

The term “lower portion of the sizing guide” refers to a part of the guide at or near the end most distal to its main body and in a downward direction away from the trochlear attachment section of the first gauge (see FIG. 1). The term “engages” as used in this context means that a gauge attaches to, or slides through, the sizing guide. For example, a gauge may traverse a slot in the sizing guide as shown in FIG. 2 (18) in a manner that allows it and the sizing guide to slide past one another. A “gauge,” in this context, is a device for measuring length, for example from the top of the trochlear groove to the top of the sizing guide.

In an especially preferred embodiment, the femoral sizing device comprises two spacer blocks, wherein one of the two spacer blocks is attached to the lower portion of the femoral sizing guide. An appropriate type of arrangement is shown in FIG. 7. In other preferred embodiments, the first gauge comprises a calibrated longitudinal arm that traverses a slot through the main body of the femoral sizing guide and this main body is in the form of a longitudinal member that engages the first gauge at about a 90 degree angle. The term “about” in this context and as used herein, means 90 degrees plus or minus up to 7%.

The femoral sizing guide should have substantially flat front and rear surfaces that extend the length of the longitudinal member and which are traversed by at least two drill holes. The term “substantially flat” means that the surface is not curved (e.g., does not deviate from being level by more than 10%, and preferably by not more than 5%). In addition, it should preferably have a flat top and bottom that are at about 90 degree angles to the front and rear.

It is also preferred that the lower portion of the sizing guide where the spacer block is attached be partly in the form of a fixed arm that extends in a direction away from the longitudinal member of the sizing guide. This arm should be at one end of the longitudinal member and the second gauge (if present) should be engaged by the femoral sizing guide at the opposite end.

In another preferred embodiment, the femoral sizing guide has a lobe at either end of the elongated member. The fixed arm extends from the lobe at one end and there is a hole or slot extending though the lobe and elongated member at the other end that may optionally be traversed by a second gauge.

In an especially preferred embodiment, the femoral sizing device comprises:
a) a femoral sizing guide comprising:
   i) a longitudinal member with a substantially flat upper surface, a bottom surface opposite to said upper surface, a front face extending the length of said longitudinal member and a rear face located opposite to said front face;
   ii) a centrally located slot extending from the upper to the lower surface of said longitudinal member;
   iii) a lobe at either end of said longitudinal member, each lobe comprising one or more drill holes;
   iv) a fixed arm extending downward from the lower end of one lobe and terminating in a section with means for attaching a spacer block;
b) a central feeler gauge comprising:
   i) a calibrated longitudinal arm that extends through the centrally located slot in said femoral sizing guide; and
   ii) a transverse member which extends in a direction away from the rear face of the femoral sizing guide
c) two spacer blocks, wherein one of said two spacer blocks is attached to the lower portion of said femoral sizing guide and wherein said two spacer blocks are joined by a handle that extends at approximately 90 degrees away from the front face of the femoral sizing device. Preferably a spacer block (or the femoral sizing guide) has pegs that can be slipped through corresponding holes on the femoral sizing guide (or spacer block) to hold the spacer in place during us. This will allow for the easy detachment of the spacer blocks from the femoral sizing guide.

It will be understood that shapes other than a square or rectangle can be used for a spacer block as this term is used herein. For example rounded or oblong spacer blocks should work just as well as square or rectangular blocks.

In another aspect, the invention includes methods of making measurements of the distal end of the femur during knee surgerymusing the device described above. Most preferred are procedures in which no measurements are made based upon the anterior condyles.

More generally, the invention encompasses all methods of preparing the distal end of the femur for resection during knee surgery in which a measurement is made between the lowest point in the trochlear groove and the lowest point of the medial posterior condyle, preferably without any measurements based upon the anterior condyles. The measurement to the lowest point of the medial posterior condyle may be extended by 1-6 mm to account for bone or cartilage loss.

The femoral sizing device and procedures described above have several advantages over many existing devices and methods. First, the measuring and tensioning of the medial flexion gap may take place essentially simultaneously, with spacer blocks or laminar spreaders being used laterally to achieve proper femoral external rotation. The pivot point for femoral external rotation is not in the center of the distal femur or on the lateral condyle, but in the center of the medial condyle. Moving the pivot point to the medial condyle results in less resection off the posterior medial condyle. Lost cartilage and bone is readily compensated for through the insertion of shims to help restore proper medial posterior condylar offset. Finally, the same instrument can be used for left and right knees.

### BRIEF DESCRIPTION OF THE DRAWINGS

The device of the present invention is illustrated in FIGS. 1-7 along with anatomical information that helps in understanding how the device may be used. The elements shown in the drawings are as follows:
1: spacer block;
2: spacer block shim designed for addition to top of a spacer block;
3: femoral sizing guide;
4: central feeler gauge;
5: lateral flexion gap;
6: cut tibial surface which will receive the tibial component of a knee prosthesis;
7: cut distal surface of the femur;
8: distal end of the femur;
9: lateral feeler gauge;
10: trochlear groove of femur;
11: drill holes;
12: posterior medial condyle;
13: posterior lateral condyle;
14: holes in arm of the femoral sizing guide which serve in attaching a spacer block;
15: longitudinal arm of central feeler gauge;
16: transverse arm of the central feeler gauge;
17. longitudinal member of femoral sizing guide;
18. slot for penetration of longitudinal arm of central feeler gauge;
19. lobes of femoral sizing guide;
20. fixed arm of femoral sizing guide that connects a spacer block to a lobe;
21. slot for penetration of lateral feeler gauge;
22. upper surface of spacer block where shims may be placed;
23. handle of spacer assembly attached to spacer block(s);
24. bottom spacer block assembly shim;
25. spacer block pegs for attachment to femoral sizing guide;

FIG. 1: FIG. 1 is a frontal view of the assembled femur sizing device. The primary elements of the device are: a femoral sizing guide (3); and a central feeler gauge (4) that engages the device. Also shown is a spacer block (1) attached to the femoral sizing guide (3). Two optional components that are shown that may, or may not, be present are a shim (2) positioned on top of the spacer block (1) and a lateral feeler gauge (9).

FIG. 2: FIG. 2 is an isolated view of the femoral sizing guide. As shown in the figure, the femoral sizing guide has longitudinal member (17) with lobes (19) on either end. Numbered holes (11) are on the front surface of one lobe of the femoral sizing guide with corresponding holes on the opposite lobe. There is a slot (18) in the top of the sizing guide through which a central feeler gauge may pass and an optional hole or slot (21) positioned over one lobe through which a second feeler gauge can pass. The femoral sizing guide has a fixed arm (20) that extends from one lobe downward at about a 90 degree angle from the longitudinal member (17) and that has holes (14) at its distal end for attaching to a spacer block.

FIG. 3: FIG. 3 is an isolated view of central and lateral feeler gauges. The central feeler gauge has a longitudinal arm (15) and a transverse arm (16). The lateral feeler gauge is shown as (9).

FIG. 4: FIG. 4 is an isolated view of a spacer block (1) with an attached shim (2). Part of the fixed arm (20) of the femoral sizing guide is also shown.

FIG. 5: FIG. 5 shows a frontal view of the femoral sizing device placed on the distal femur (8), which has been cut (7) by a surgeon. During use, the fixed arm of the femoral sizing guide (3) is placed on the front surface of the posterior medial condyle (12) and serves to position an attached spacer block (1) beneath this condyle and on top of the tibia (6). A shim (2) is shown on the top of the spacer arm that adjusts the height of the spacer block so that the bottom of the condyle is contacted. On the side of the femoral sizing guide opposite to the fixed arm, there is a lateral feeler gauge (9) that is optionally present and that may be used to make measurements of cuts in the area of the posterior lateral condyle (13) located over the lateral flexion gap (5). Also shown in the figure, is the central feeler gauge (4) that extends through a slot in the femoral sizing guide and that is suspended by its transverse arm from the lowest part of the trochlear groove (10) of femur. The word “RIGHT” on the front of the femoral sizing guide indicates the front face that should be facing the surgeon when it is the right knee that is being operated on. In order to use the device on the left femur, the central feeler gauge and spacer block can be detached from the femoral sizing guide and then reinserted in a proper orientation after the device is rotated 180 degrees.

FIG. 6: FIG. 6 shows a spacer assembly. This has a handle (23) that is attached to spacer blocks (1) and that can be used to attach these spacer bock(s) to (or remove them from) the femoral sizing guide during operation of the device. The assembly depicted shows a preferred embodiment in which there are two spacer blocks. Also shown in the figure is a bottom spacer assembly shim (24) which interconnects with the bottom of the assembly to help adjust the height of the spacer blocks. Height can also be adjusted by attaching shims to the top surface of the spacer blocks (22) which are designed to engage or interlock the shims in order to hold them in place.

FIG. 7: FIG. 7 shows a spacer block assembly with two spacer blocks attached to the femoral sizing guide (3). In this drawing, shims (2) are shown in place on the spacer blocks (1). Pegs (25) can be seen which are slipped through holes (14) in the fixed arm (20) of the femoral sizing guide. The handle (23) of the spacer block assembly can be used to conveniently attach or remove the spacer blocks (1) from the femoral sizing guide (3). The central feeler gauge (4) with its transverse arm (16) is also shown.

### DETAILED DESCRIPTION OF THE INVENTION

A. Femoral Sizing Device

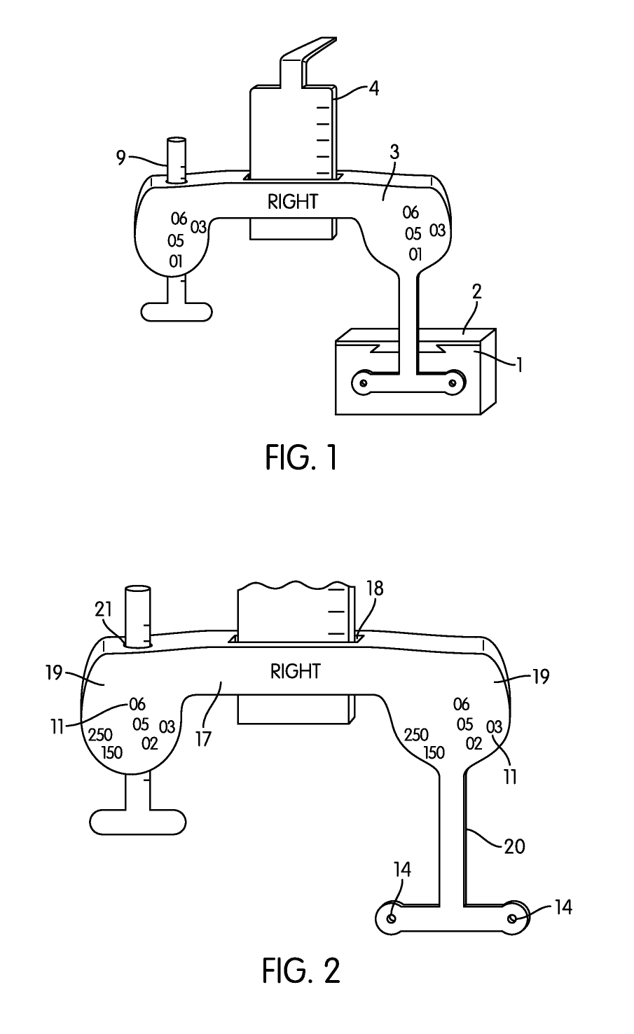

The present invention is directed to a device which can be used in making measurements for a knee resection. The device may be made using standard materials for surgical devices and will include a femoral sizing guide and a central feeler gauge. When completely assembled for use, it will also include at least one, and preferably two, spacer blocks. Having two blocks instead of one, makes the device more stable during use. The general characteristics of these components and the way that they come together to form the sizing device may be understood by reference to FIGS. 1-7.

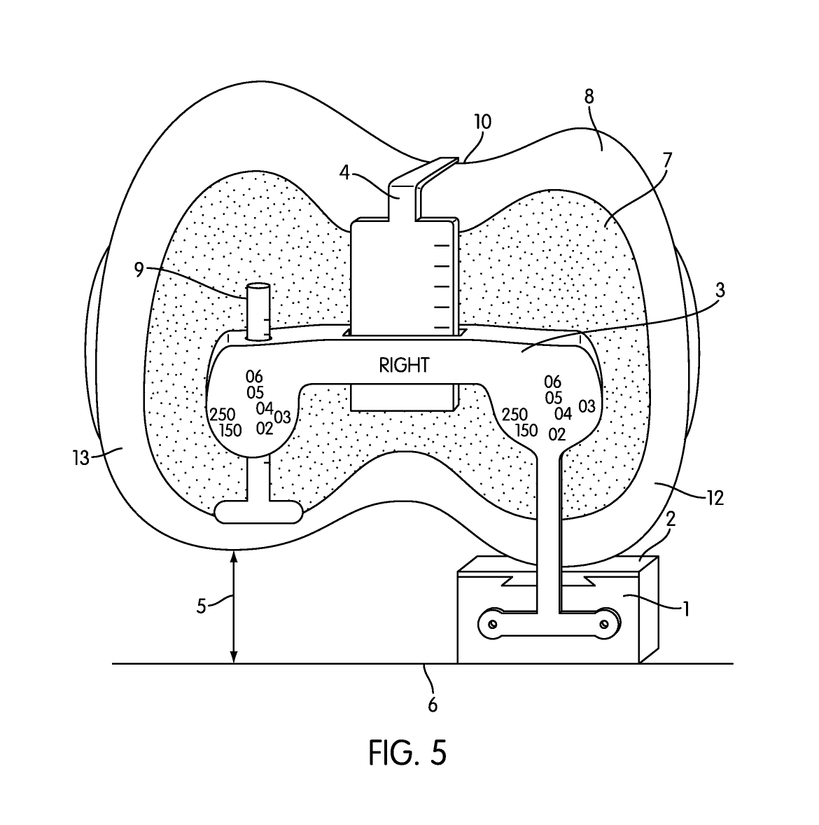

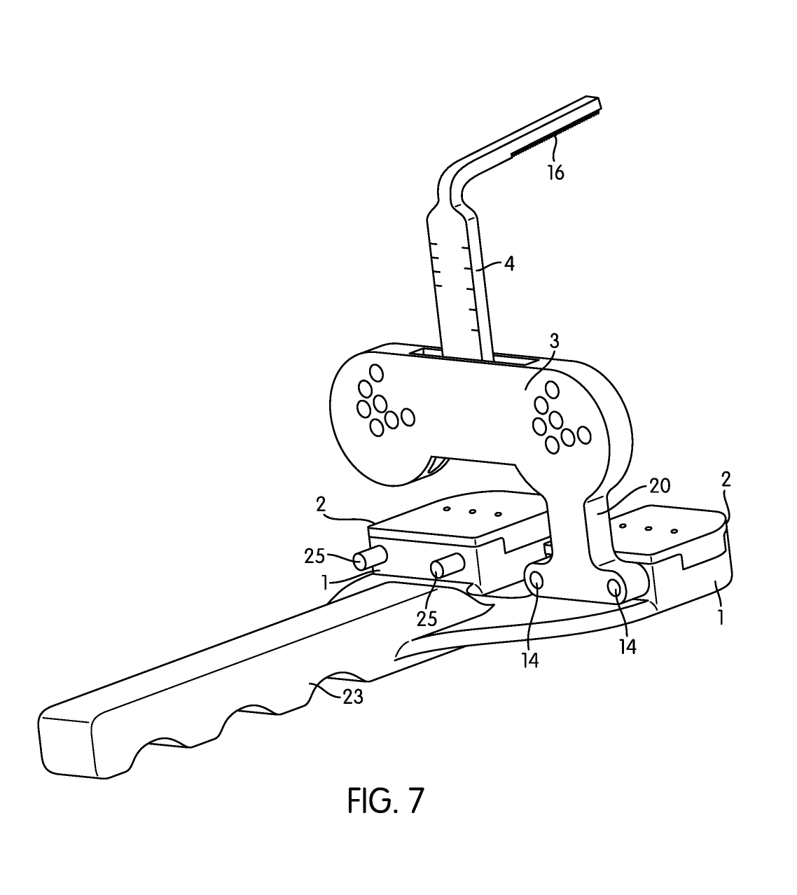

The femoral sizing guide (FIGS. 1 and 7, (3)) is shown in the figures as having an elongated longitudinal member (FIG. 2, (17)) that approximately spans the centers of the posterior condyles (FIGS. 5, (12 and 13)) on the distal end of a femur (FIG. 5, (8)) that has been cut (FIG. 5, (7)). The front of the sizing guide faces forward in FIGS. 1, 2 and 5 and has a substantially flat surface. Opposite this is a rear surface that is also substantially flat and that rests against the cut surface (FIG. 5, (7)) of the femur (FIG. 5 (8)). Lobes (FIG. 2, (19)) are preferably located at either end of the sizing guide with various numbered holes (FIG. 2, (11)). These lobes may be either essentially circular or somewhat more oval in shape. Once the device has been properly positioned, the holes will serve as guides for drilling holes that will subsequently be used for positioning a chamfer or other device for cutting the femoral bone.

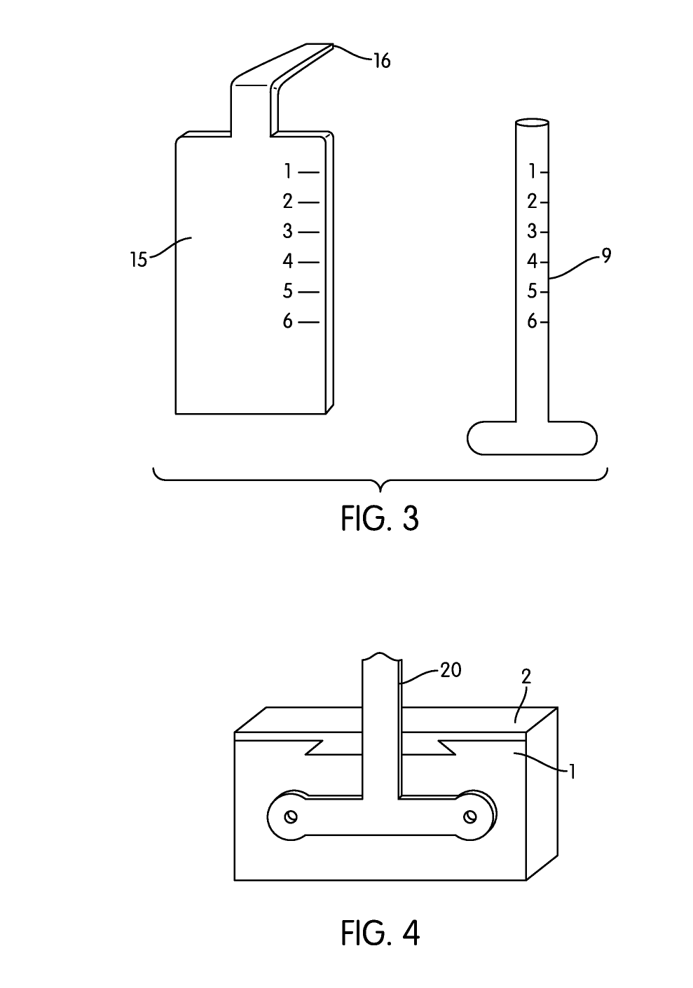

Centrally located on the top surface of longitudinal member of the drilling guide (FIG. 2, (17)) is a slot (FIG. 2, (18)). The longitudinal arm (FIG. 3, (15)) of a centrally located feeler gauge (FIG. 1, (4)) traverses the slot (FIG. 2, (18)) at approximately an angle of 90°) and has calibrations for making measurements. The central feeler gauge moves, or can be moved, easily through the slot (FIG. 2, (18)) and the calibrations are used to determine the relative location of the longitudinal member (FIG. 2, (17)) of the drilling guide.

At the top of the longitudinal member of central feeler gauge (FIG. 3, (15)), there is a transverse member (FIGS. 3 and 7, (16)) which extends at approximately a 80-130° (preferably 80-110°) angle away from the rear face of the femoral sizing guide. As shown in FIG. 5, the purpose of this member is to suspend the sizing device from the lowest part of the trochlear grove (10) of the femur (8). If desired, there may be sufficient contact between the longitudinal arm of the central feeler gauge (FIG. 3, (15)) and the longitudinal arm of the femoral sizing guide (FIG. 2, (17)) within the slot (FIG. 2, (18)) to prevent the central feeler gauge from slipping through due simply to the weight of the sizing guide and other components of the device. Falling may be prevented due to friction or due to contacts between the gauge and the sizing guide created by a roller or ratchet mechanism. Alternatively, there may be a device for locking the gauge at a particular position and releasing it when an operator wants to move it.

Extending from one lobe of the sizing guide is a fixed arm (FIGS. 2 and 7, (20)) that extends down at about a 90° angle relative to the elongated member of the femoral sizing guide (FIG. 2, (17)). This terminates in a region with means (FIGS. 2 and 7, (14)) for connecting a spacer block (FIGS. 1, 4, and 7 (1)) to the femoral sizing guide. Any means of attachment may be used including screws, pins, clasps, etc. However, the preferred means are pegs (FIG. 7 (25)) on the spacer blocks (FIG. 7 (2)) that slip into corresponding holes (FIG. 7 (14)) on the fixed arm (FIG. 7 (20)) of the femoral sizing guide (FIG. 7 (3)).

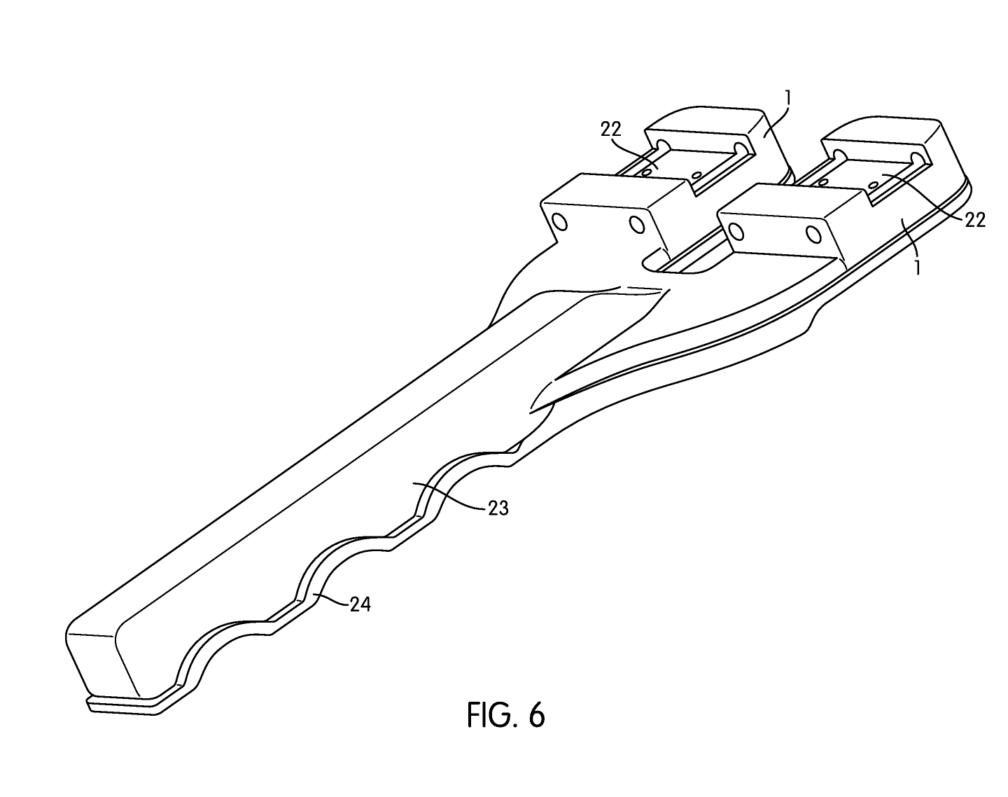

The spacer block (FIG. 4 (1)) may be present at different thicknesses and these thicknesses should be equivalent to the composite tibial component of the implant plus 1-2 mm to allow for joint laxity. Preferably, there are two spacer blocks (FIG. 6 (2)) that are joined together by a handle (FIG. 6 (23)) to form a spacer block assembly. This arrangement allows a surgeon to easily attach and detach the spacer blocks to the femoral sizing guide and the presence of two spacer blocks, as opposed to one, tends to make the device more stable when being used for making measurements. A bottom spacer block assembly shim (FIG. 6 (24)) may be used together with shims (FIGS. 4 and 7 (2)) placed on the upper surface of spacer blocks (FIG. 6 (22)) to adjust the height of spacer blocks.

As shown in FIG. 5, during an operation, the spacer block is positioned so that it is resting on the surface of the tibia (FIG. 5 (6)). One or more shims (FIGS. 4 and 7, (2)) may be added to the upper surface of spacer blocks (FIG. 6 (22)), thereby increasing its thickness to fill the medial flexion gap while keeping the distance between the pegs (FIG. 2, (14)) attaching the spacer block and the drill holes (FIG. 2 (11)) constant. These shims may come in various thicknesses in 1 mm increments to estimate the total amount of cartilage and bone loss off the posterior medial femoral condyle and, as mentioned above, may optionally be used with a bottom spacer block assembly shim (FIG. 6 (24)). The purpose is to anatomically restore the posterior condylar offset. Given a cartilage thickness of 2-3 mm this amount is added to the amount of bone loss and then the selected shim is attached to the spacer-block (FIG. 4, (1)), using any type of common attachment method (e.g., FIG. 4 shows attachment using a dovetail joint).

The front and back of the femoral sizing guide are symmetric so the device can be used for either the left or the right knee. The central feeler gauge (FIGS. 3, 5 and 7 (4)) is removable and can be flipped to function on either side. This feeler gauge slides up and down and helps measure the anterior posterior (“AP”) dimension from the most inferior posterior condyle to the top of the lowest point of the trochlear groove. The central feeler gauge may be constructed to slide medially and laterally in a slot (FIG. 2 (18)) of the femoral sizing guide to find the lowest point of the trochlear groove.

In order to adjust the lateral tendon and internally rotate the femur, the surgeon inserts either spacer-blocks, a laminar spreader or other device (e.g., a calibrated tensiometer) into the lateral flexion gap (FIG. 5 (5)). The tension is subject to surgeon's preference. The more distraction in the flexion gap, the more external rotation and less resection off the lateral femoral component will be achieved.

In order to help determine the amount of resection needed to account for the selected lateral flexion gap, the femoral sizing device may include a lateral feeler gauge (FIGS. 1 and 3 (9)), which slides through a hole or slot (FIG. 2 (21)) in the femoral sizing guide in essentially the same way as the central feeler gauge. The lateral feeler gauge will typically be calibrated.

As will be recognized by those of skill in the art, many insubstantial variations may be introduced into the design of the device described above without changing its operation. For example, the exact size and shape of the femoral sizing guide is not critical, the feeler gauges may be altered in ways that do not interfere with the measurements being made and the blocks and shims used in sizing the medial flexion gap may be replaced by equivalent spacers with different shapes and designs. Insubstantial differences of these types are part of the invention.

B. Sizing Methodology Using the Femoral Sizing Device

The device described above has been designed for use in total knee replacement procedures (described more fully below) and provides two important measurements. One is the distance between the center of the femoral log to the most inferior point on the posterior femoral condyle. Depending on the prosthesis chosen, this distance may vary depending on prosthesis size or may be kept constant (Smith and Nephew, Genesis II, Biomet Vanguard Total Knee). If the distance remains constant throughout all sizes, there will be only one drill hole (11) for the attachment of the femoral cutting block. If this distance varies (Depuy PFC Sigma) there will be multiple drill holes. The drill holes are markers for insertion of a chamfer cutting block for the distal femur.

The AP distance between the most inferior point off the posterior condyle and the deepest portion of the trochlear groove is important for restoring femoropatellar kinematics. The device disclosed herein does not reference off the anterior femoral cortex. The distance between the anterior cortex and the deepest portion of the trochlear groove (trochlear offset) varies. Thus, if the reference for AP sizing is related to the anterior cortex, the femoropatellar level arm may decrease and will change knee kinematics. This may be even worse if the distal femoral cut is proximalized as is recommended in some surgical manuals.

The patella is a crucial part of the reverse screw home mechanism. The original patella thickness should be reconstructed and its thickness restored. By leaving the lateral side slightly looser in flexion and by restoring the trochlear groove, the patella can push the lateral femoral condyle more posteriorly with increased flexion. This, in combination with an appropriately tensioned medial cruciate ligament (MCL) and posterior cruciate ligament (PCL) facilitates lateral roll-back. The functional restoration of the balance between the patella in the front, the MCL in the posterior medial corner and the PCL in the center of the posterior tibia depends upon these structures,-where they are positioned in 3D and how they are tensioned. It is also important to restore the joint line of the distal femur in extension and flexion. Specifically the restoration of the posterior medial condylar offset is important to achieve closer to normal knee kinematics. If one element is too taut or too loose the medial condyle does not remain more or less stable on the medial tibial condyle and tibial internal rotation with increased flexion and lateral condylar roll-back is not observed. The femoral sizing device described herein reconstructs the geometry of the medial J-curve and the trochlear groove and restores ligament tensions on the medial side without releases of either the PCL or MCL.

C. Surgical Techniques

A surgical technique for total knee replacement may be used that follows the principles of measured resection on the medial side and combines it with a femoral and tibial independent restoration of the joint line. The method uses a balancing gap technique to increase femoral component external rotation, and preserves the original tension of the posterior cruciate ligament by not releasing it at all. Unlike many procedures the total knee is balanced first in flexion, not extension.

The tibial component is positioned 90 degrees perpendicular to the tibial mechanical axis and the femoral component is positioned between 3 to 7 degrees of valgus along the coronal femoral mechanical axis. The reduction of the amount of valgus for femoral placement is necessary to correct for the symmetric geometry of the femoral component and the increased lateral tibial resection secondary due to a reduction of the anatomic tibial varus from 87 degrees to 90 degrees.

The geometry of the trochlea is reconstructed. Normally, the anterior femoral cut is flush with the anterior cortex, removing more trochlea bone stock than is replaced with the femoral component. This decreases the femoropatellar level arm and may increase quadriceps strength e.g., in walking stairs. The present technique follows the principle of resecting only the amount of trochlea equal to the component thickness and the deepest point of the anatomic trochlea is not greatly changed in its position after implant insertion.

The anterior cut is performed in a more flexed position to reduce the risk of notching the medial anterior cortex by 7 to 10 degrees. This applies for PCL retaining implants only.

To reconstruct trochlea geometry, the distal femoral medial joint surface geometry is restored. Traditionally, 7 to 9 mm of bone are resected off the affected medial condyle distally. In the present technique, bone loss is estimated in mm and about 3 mm of cartilage thickness are added. This amount is subtracted from the implant thickness and in most cases only 3-5 mm will be taken off the distal medial condyle. The distal coronal valgus angle is reduced to 3-7 degrees. By restoring the geometry of the distal medial condyle, mid-flexion instability is eliminated.

Tibial resection is based on the same basic principles. The tibia is exposed, osteophytes are resected and, in mild varus, releases to correct deformities are not necessary. The medial sleeve is preserved and only the tibio-meniscal ligament is taken down 3-4 mm below the jointline to protect the medial collateral ligament (MCL). In varus knees, the amount of tibial resection follows the concept of restoring the joint line and the original tension of the MCL.

The amount resected on the lateral side is based on the concept of cutting the tibia at 90 degrees perpendicular to the tibial mechanical axis. The assumed amount, x, of bone in mm, lost at the deepest point of the tibia plus 3 mm of cartilage is subtracted from the tibial component thickness and equals the amount of bone needed to be resected off the medial tibia.

Next, the flexion gap is measured. Using spacer-blocks in various thicknesses, calibrated laminar spreaders or a tensiometer, the flexion gap is measured at 90 degrees flexion in mm medially. The gap represents the tibial composite implant thickness. The geometry and thickness of the posterior condyle is just restored by resecting the amount needed to replace it with the implant thickness. Most implants are between 7-9 mm thick posteriorly. So, if the posterior condyle lost, e.g., 1 mm of cartilage, 1 mm less than the implant thickness is removed off the posterior condyle to ensure appropriate reconstruction of the posterior condylar offset.

The knee is then brought into extension and balanced. If the extension gap is too tight, more bone is resected off the distal femur until the extension gap is equally balanced as compared to the flexion gap. Since much less bone is resected off the distal femur, a too tight extension gap will only occur if the tibial resection is insufficient.

D. Femoral Rotation

With the knee in 90 degrees of flexion, medial and lateral gaps are tensioned with spacer blocks, laminar spreaders, a tensiometer or any other mechanical device. Under tension, the femur is sized using anterior and posterior references. Anteriorly, the trochlea is marked parallel to the tibia. If the trochlea groove is bare bone, the joint surface is reconstructed by cutting less off as compared to the thickness in the trochlea.

Posteriorly, the resection is calculated medially only, depending on the total amount of cartilage and bone loss. The posterior resection is more important than the anterior resection, i.e., the posterior condyle has to be restored. If too much bone is resected, the flexion gap will be too loose and if too little is resected as compared to the component thickness, the flexion gap will be too tight. If the anterior-posterior sizing is in between sizes the smaller component is chosen, but the appropriate amount is still resected off the posterior condyle. In other words, the posterior thickness is restored and more bone is taken off anteriorly.

Resecting more bone anteriorly is possible due to the fact that the present technique does not cut flush down to the anterior cortex, but in 7-10 degrees of flexion. Also, since the anterior cut is more flexed as compared with traditional techniques, notching is less likely. Sizing is based only on the geometry of the medial condyle.

The flexion gap is distracted with equal force medially and laterally. A line parallel to the tibial cut is marked with a pen on the femur (or tools can be used) increasing the tibial cut to find the position for the femoral cutting block. If the difference between both medial and lateral gaps is more than the implant thickness, ligament structures laterally are too loose. It is recommended to resect at least 1-4 mm of bone off the posterior lateral condyle. If the medial flexion gap opens too much, the medial collateral ligament is insufficient and this technique cannot be performed.

Most systems will have a femoral chamfer cutting block with two central pins: one going into the medial condyle and the other one into the lateral condyle. The position of the medial pin should remain fixed to preserve the amount of bone resected off the posterior condyle. If the lateral pin is not equally distanced to the tibial cutting plane, the pin is moved closer to the tibial plane, until medial and lateral pin have the same distance to the tibial cutting plane. This externally rotates the femoral position and balances the flexion gap.

Most of the time, less bone is taken off the posterior lateral condyle due to the external rotation of the femoral component. Since, in the present technique, the axis of external rotation is preferably positioned in the center of the medial condyle, the amount of resection off the posterior medial condyle does not change.

Femoral cuts are now completed. After removal of all residual meniscal tissue and osteophytes, trial components are placed and the range of motion and balancing is documented. Positioning of the femoral component is marked, the tibial trial removed and using special off-set osteotomes, all osteophytes around the prosthetic edges of the posterior condyles are removed.

Final preparation of femur and tibia, using punches or drills for the femoral lugs and tibial stem are completed in standard fashion. The positioning of the tibial tray follows published techniques (Stiehl, et al., J. Bone Joint Surg. Br. 77:884 (1995); Dennis, et al., Clin. Orthop. 428:180 (2004); Stiehl, et al., Clin. Orthop. 365:139 (1999); Banks, et al., J. Arthroplasty 3:297 (1997); Bertin, et al., J. Arthroplasty 17:1040 (2002)). Components are cemented in standard fashion. After polymerization is completed, residual bone cement is removed and the final insert placed.

If the above technique is properly performed, several characteristics should be present in the reconstructed knee after surgery. First, in extension between 0 and 15 degrees, the medial opening under valgus stress should be about 1 mm, and lateral under varus stress should be between 1-2 mm. At 20 to 50 degrees, there should be no substantial mid-flexion instability and the knee should open only 1-2 mm. At 90 degrees of flexion, the tibia should internally rotate and the lateral condyle roll backwards. Medially, the condyle should remain in the center of the medial tibial condyle. The PCL should not be tight and there should be no paradoxical roll forward. The knee should flex against gravity easily and provide good flexion.

The technique should work for valgus knees in the same fashion, but resection of bone off the tibia plateau medially should be reduced by about 2 mm, taking only 6 mm off laterally and medially.

All references cited herein are fully incorporated by reference. Having now fully described the invention, it will be understood by one of skill in the art that the invention may be performed within a wide and equivalent range of conditions, parameters, and the like, without affecting the spirit or scope of the invention or any embodiment thereof

1. A femoral sizing device comprising:
a) a first feeler gauge with a trochlear attachment for suspending said first feeler gauge from the lowest point of the trochlear groove of the femur; and
b) a femoral sizing guide, comprising a main body which engages said first feeler gauge, and which includes means for attaching a spacer block and for positioning said spacer block beneath a posterior medial condyle of a femur when said femoral sizing guide engages said first feeler gauge.

2. The femoral sizing device of claim 1, further comprising a fixed arm for positioning said spacer block beneath said posterior medial condyle, wherein said fixed arm is located at one end of a longitudinal member of said sizing guide and extends in a direction away from said longitudinal member.

3. The femoral sizing device of claim 2, wherein said means for attaching said spacer block is selected from the group consisting of: pegs, holes, screws, pins, clamps or clasps.

4. A femoral sizing device comprising:
a) a first gauge with a trochlear attachment section for suspending said first gauge from the lowest point of the trochlear groove of the femur;
b) a femoral sizing guide, comprising a main body which engages said first gauge; and
c) at least one spacer block, attached to the lower portion of said femoral sizing guide.

5. The femoral sizing device of claim 4, wherein said femoral sizing device comprises two spacer blocks, wherein one of said two spacer blocks is attached to the lower portion of said femoral sizing guide.

6. The femoral sizing device of claim 5, wherein, said first gauge comprises a calibrated longitudinal arm that traverses a slot through the main body of said femoral sizing guide.

7. The femoral sizing device of claim 5, wherein the main body of said femoral sizing guide comprises a longitudinal member that engages said first gauge at about a 90 degree angle.

8. The femoral sizing device of claim 7, wherein said femoral sizing guide has a substantially flat front and rear that extend the length of said longitudinal member and that are traversed by one or more drill holes.

9. The femoral sizing device of claim 8, wherein said femoral sizing guide has a flat top and a bottom that are at about 90 degree angles to said front and said rear of the sizing guide.

10. The femoral sizing device of claim 9, wherein there is a slot that extends through the top and bottom of said femoral sizing guide, wherein said slot is traversed by the calibrated longitudinal arm of said first gauge.

11. The femoral sizing device of claim 10, wherein the lower portion of said femoral sizing guide includes, in part, a fixed arm that is located at one end of the longitudinal member of said sizing guide and that extends in a direction away from said longitudinal member.

12. The femoral sizing device of claim 11, wherein the end of said fixed arm distal to the longitudinal member of said femoral sizing guide is attached to a spacer block.

13. The femoral sizing device of claim 12, wherein said femur femoral sizing guide engages a second gauge at the end of said longitudinal member that is opposite to the end with said fixed arm.

14. The femoral sizing device of claim 13, wherein:
a) said femoral sizing guide has a lobe at either end of said elongated member;
b) said fixed arm extends from the lobe on one end; and
c) there is a hole or slot extending though the lobe and elongated member at the other end that is traversed by said second gauge.

15. The femoral sizing device of claim 14, wherein the thickness of said spacer block is increased by one or more shims added to the surface of said spacer block.

16. A femoral sizing device comprising:
a) a femoral sizing guide comprising:
i) a longitudinal member with a substantially flat upper surface, a bottom surface opposite to said upper surface, a front face extending the length of said longitudinal member and a rear face located opposite to said front face;
ii) a centrally located slot extending from the upper to the lower surface of said longitudinal member;
iii) a lobe at either end of said longitudinal member, each lobe comprising one or more drill holes;
iv) a fixed arm extending downward from the lower end of one lobe and terminating in a section with means for attaching a spacer block;
b) a central feeler gauge comprising:
i) a calibrated longitudinal arm that extends through the centrally located slot in said femoral sizing guide; and
ii) a transverse member which extends in a direction away from the rear face of the femoral sizing guide
c) two spacer blocks, wherein one of said two spacer blocks is attached to the lower portion of said femoral sizing guide and wherein said two spacer blocks are joined by a handle that extends at approximately 90 degrees away from the front face of the femoral sizing device.

17. The device of claim 16, wherein either
a) said at least one of said two spacer blocks comprises pegs that slip into corresponding holes on the femoral sizing guide to attach the spacer block and femoral sizing guide together; or
b) said femoral sizing guide comprises pegs that slip into corresponding holes on at least one of said two spacer blocks to attach the spacer block and femoral sizing guide together.

18. The device of claim 17, wherein a lateral slot or hole extends from the upper to the lower surface of said longitudinal member through the bottom of the lobe on the end of said longitudinal member opposite the end with said fixed arm.

19-22. (canceled)
